(19) United States
(12) Patent Application Publication
(10) Pub. No.: US 2025/0084358 A1
(43) Pub. Date: Mar. 13, 2025

(54) PRODUCTS OF MANUFACTURE FOR CUL TURING OR MAINTAINING NORMAL, PRE-CANCER AND CANCER STEM AND PROGENITOR

(71) Applicant: The Regents of the University of California, Oakland, CA (US)

(72) Inventors: Catriona JAMIESON, La Jolla, CA (US); Luisa LADEL, La Jolla, CA (US); JESSICA PHAM, La Jolla, CA (US); Isabelle OLIVER, La Jolla, CA (US); Larisa BALAIAN, La Jolla, CA (US); Christina WU, La Jolla, CA (US); Wenxue MA, La Jolla, CA (US); Ludmil ALEXANDROV, La Jolla, CA (US); Shuvro NANDI, La Jolla, CA (US); Thomas WHISENANT, La Jolla, CA (US)

(21) Appl. No.: 18/729,810

(22) PCT Filed: Jan. 12, 2023
(86) PCT No.: PCT/US2023/010679

§ 371 Date: Jul. 17, 2024

Related U.S. Application Data
(60) Provisional application No. 63/298,936, filed on Jan. 12, 2022.

(51) Int. Cl. C12M 1/04; C12M 1/00; C12N 5/0789

(52) U.S. Cl. CPC C12M 23/24; C12M 23/14; C12M 23/40; C12M 29/12; C12N 5/0647; C12N 2513/00

(57) ABSTRACT

In alternative embodiments, provided are biosensing bioreactors that detect normal, pre-cancer and cancer stem cells. In alternative embodiments, provided are bioreactors for primary human hematopoietic stem and progenitor cell culture, manipulation and maintenance, which can comprise micro-peristaltic pumps to recapitulate blood flow. In alternative embodiments, provided are products of manufacture in the form of a bioreactor, or a nanobioreactor, or a customized cell culture bag with a defined three dimensional (3D) stromal microenvironment, and kits comprising them, and methods for making and using them. In alternative embodiments, the products of manufacture as provided herein, including the bioreactor, nanobioreactor, bag, customized cell culture bag and kits as provided herein, are used to and are capable of culturing, maintaining or supporting the culture of, and/or differentiating hematopoietic stem cells, including human hematopoietic stem cells, isolated from bone marrow or blood donors.

### RELATED APPLICATIONS

This Patent Convention Treaty (PCT) International Application claims the benefit of priority under 35 U.S.C. § 119(e) of U.S. Provisional Application No. 63/298,936, filed Jan. 12, 2022. The aforementioned application is expressly incorporated herein by reference in its entirety and for all purposes. All publications, patents, patent applications cited herein are hereby expressly incorporated by reference for all purposes.

### TECHNICAL FIELD

This invention generally relates to biosensing bioreactors and cancer biology. In alternative embodiments, provided are biosensing bioreactors that detect normal, pre-cancer and cancer stem cells. In alternative embodiments, provided are bioreactors nanobioreactors, bags or culture bags for primary human hematopoietic stem and progenitor cell culture, manipulation, differentiation and maintenance, which can comprise micro-peristaltic pumps to circulate fluids and simulate or recapitulate blood flow. In alternative embodiments, provided are products of manufacture in the form of a bioreactor, or a nanobioreactor, bag, or a customized cell culture bag with a defined three dimensional (3D) stromal microenvironment, and kits comprising them, and methods for making and using them, and optionally also comprising bioluminescent and fluorescent lentiviral reporter vectors capable of quantifying normal, pre-cancer and cancer stem cell function, including FUCCI2BL cell cycle reporter, an ADAR1-nanoluciferase-GFP reporter self-renewal reporter and an RFP/GFP splicing reporter. In alternative embodiments, the products of manufacture as provided herein, including the bioreactor, nanobioreactor, customized cell culture bag and kits as provided herein, are used to and are capable of culturing, maintaining or supporting the culture of, and/or differentiating hematopoietic stem cells, including human hematopoietic stem cells, isolated from bone marrow or blood donors. In alternative embodiments, the products of manufacture as provided herein, including the bioreactor, nanobioreactor, customized cell culture bag and kits as provided herein, are used to and are capable of supporting or maintaining a primary human hematopoietic stem cell culture.

### BACKGROUND

While stem cells play a vital role during embryonic and fetal development, they also maintain adult tissue integrity and can be mobilized in response to injury to repair and regenerate tissues. Stem Cells are defined functionally based on their capacity to self-renew (divide without differentiating), differentiate into tissue-specific progenitors and become dormant in protective microenvironments. This is where stem cells typically reside in the body in homeostatic and healthy states. These microenvironments maintain highly specific and tightly regulated conditions and are often referred to as “niche”. The hematopoietic stem cell niche lies within the highly vascularized, trabecular bone marrow.

### SUMMARY

In alternative embodiments, provided are products of manufacture (which can be in the form of a bioreactor, or a nanobioreactor, or a customized cell culture bag with a defined three dimensional (3D) stromal microenvironment) for cell culture, wherein optionally the cells are stem cells or primary human hematopoietic stem cells and/or progenitor cell cultures, and/or for the maintenance, expansion and/or differentiation of these cell cultures, comprising:
a container, enclosure or bag having gas-permeable (and optionally, liquid impermeable or liquid impervious) walls or sides, and a three dimensional (3D) sponge matrix or a sponge-like material contained within the container, enclosure or bag,
wherein:
the sponge matrix or sponge-like material is infused with a cell culture media and a mixture of cells comprising stem cells and/or bone marrow matrix cells,
the container, enclosure or bag interior is sterile, and
the container, enclosure or bag comprises at least one liquid or cell input port, or at least two liquid or cell input ports.

In alternative embodiments of products of manufacture as provided herein:
the products of manufacture (which also can be called a bag or a container, or is in the form of a bioreactor, or a nanobioreactor, or a customized cell culture bag with a defined three dimensional (3D) stromal microenvironment) is fabricated as a gas-permeable (and optionally, liquid impermeable or liquid impervious) cell culture bag or container, or equivalent;
the container, enclosure or bag is fabricated as a gas-permeable (and optionally, liquid impermeable or liquid impervious) cell culture environment, or bag;
the container, enclosure or bag comprises at least two liquid or cell input ports, one out port and one in port, optionally a micro-peristaltic pump for circulating liquid inside the container, enclosure or bag is operably connected to the in port and the out port,
the mixture of cells comprising stem cells, or human stem cells, bone marrow matrix cells, or human bone marrow matrix cells, or cancer stem cells, or human cancer stem cells, or organoid cells, or any combinations thereof;
and optionally the mixture of cells comprises CD34+ donor-derived human hematopoietic cells (HSCs), or human CD34+ donor-derived human hematopoietic cells (HSCs) or pre-cancer stem cells or cancer stem cells or cancer cell lines lentivirally transduced with biosensing reporters of stem cell activity (for example, comprising FUCCI2BL, ADAR1 and/or a splicing reporter), or any combinations thereof;
the sponge or sponge-like material comprises:
   an absorbable gelatin sponge (optionally an absorbable human or porcine gelatin sponge),
   a solubilized or reconstituted basement membrane matrix,
   a solubilized or reconstituted laminin/collagen IV-rich basement membrane extracellular matrix, optionally, MATRIGEL™ (Corning Life Sciences) or GELTREX™ (ThermoFisher Scientific),
   a compressed sponge with adsorbable gelatin, optionally GELFOAM™ (Pfizer),
   a demineralized cancellous sponge (Berkeley Advanced Biomaterials (BAB), Berkeley CA), or VIASORB™ (Globus Medical, Audubon, PA); and/or
   a hydrogel-based macroporous sponge or porous hydroxipropylcellulose (HPC) scaffold, optionally CELLUSPONGE™, CELLUSPONGE-GAL™ , or CELLUSPONGE-COL™ (Bio-Bybios), optionally a cancellous (or trabecular) bone sponge, for example, CANCELLOUS SPONGE™ (VMI Medical);
the sponge or sponge-like material further comprises: a demineralized cancellous bone matrix sponge, or demineralized bone matrix (DBM) components, optionally comprising OSTEOSPONGE™ (Xtant Medical); and/or
between about 10⁶ to about 10¹⁰ cells, or between about 10⁵ to about 10¹¹ cells, between about 10⁵ to about 10¹² cells, are placed into, or cultured in, the product of manufacture.

In alternative embodiments, products of manufacture as provided herein further comprise a detectable vector or reporter, wherein optionally the detectable vector or reporter is inserted (for example, optionally transduced or transfected) in a cell (or if the vector is a viral vector or a reporter is contained in a viral vector, the viral vector can infect the cells), or substantially most of the cells in the product of manufacture, or all of the cells in the product of manufacture, and optionally comprising a bioluminescent and/or a fluorescent vector, optionally comprising a bioluminescent and/or a fluorescent lentiviral reporter vector, wherein optionally the bioluminescent and/or a fluorescent vector is capable of quantifying cells or cell function, optionally capable of quantifying normal, pre-cancer and/or cancer stem cell function, optionally the detectable vector or report comprises a FUCCI2BL cell cycle reporter, an ADAR1-nanoluciferase-GFP reporter self-renewal reporter and/or an RFP/GFP splicing reporter.

In alternative embodiments, provided are methods for culturing or maintaining or differentiating a broad spectrum of stem cells, including tissue-specific organoids and malignant organoids representing solid tumors, like breast cancer thereby promoting the plurality of stem cells in a product of manufacture as provided herein.

In alternative embodiments, provided are methods for producing an organoid, comprising incubating in a product of manufacture as provided herein a plurality of stem cells, optionally further comprising cytokines and Matrigel or bone marrow stromal cells capable of differentiating the tissue specific stem cells and/or pre-cancer or cancer stem cells.

In alternative embodiments, provided are kits comprising a product of manufacture as provided herein, optionally further comprising instructions for practicing a method as provided herein.

In alternative embodiments, provided are uses of a product of manufacture as provided herein, for primary human hematopoietic stem cell culture, or maintaining stem cells, or for differentiating cells, or for producing an organoid.

In alternative embodiments, provided are products of manufacture or kit for use for primary human hematopoietic stem cell culture, or for use in maintaining stem cells, or for differentiating cells, or for producing an organoid, wherein the product of manufacture is a product of manufacture as provided herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A illustrates images of exemplary products of manufacture for primary human hematopoietic stem and progenitor cell culture and/or maintenance, also called a bioreactor, a nanobioreactor, a customized cell culture bag or a bioreactor bag, which comprises sponges, and the bags are filled with cell culture media: Left panel: exemplary 30 ml cell culture bag with two-port system; Right panel: exemplary 7 ml cell culture bag with one-port system.

FIG. 1B illustrates representative images of A549 adherent cells in (for example, the cells are being maintained and/or cultured in) an exemplary product of manufacture as provided herein, the cells transduced with a GFP fluorescent marker; and the images confirm adherent cell line viability and imaging capability using brightfield and fluorescence through the exemplary products of manufacture (which can be called “bags”), which include sponge material; images were taken with 4× objective.

FIG. 1C illustrates representative images of TF1a suspension cells in (for example, the cells are being maintained and/or cultured in) an exemplary product of manufacture as provided herein, the cells transduced with mVenus and mCherry fluorescent markers, which confirm suspension cell line viability and imaging capability; brightfield and fluorescence through bag and sponge was used; images were taken with 20× objective.

FIG. 1D illustrates representative images of human hematopoietic stem cells, which were isolated from donor bone marrow 2 weeks after seeding, where the cells are in (for example, the cells are being maintained and/or cultured in) an exemplary product of manufacture as provided herein, and images confirm primary stem cell viability and attachment of the cells to a sponge matrix and proliferation of the within the product of manufacture, thus making the product of manufacture a nanobioreactor; images were taken with 20× objective.

FIG. 1E illustrates representative images of human hematopoietic stem cells isolated from donor bone marrow and transduced with mVenus fluorescent marker 2 weeks after seeding, where the cells are in (for example, the cells are being maintained and/or cultured in) an exemplary product of manufacture as provided herein, and images confirm primary stem cell viability and imaging capability of brightfield and fluorescence through bag and sponge. Images taken with 20× objective.

FIG. 1F illustrates representative images of human hematopoietic stem cells isolated from donor bone marrow and transduced with mVenus and mCherry fluorescent markers 4 weeks after seeding, where the cells are in (for example, the cells are being maintained and/or cultured in) an exemplary product of manufacture as provided herein, and the images confirm primary stem cell viability in the product of manufacture (or bioreactor) after 4 weeks; and imaging capability used brightfield and fluorescence through the product of manufacture (or exemplary bag and sponge; wherein the images were taken with 10× objective.

FIG. 2 schematically illustrates an exemplary modeling of a cell or culture niche, or bone marrow niche, for an exemplary product of manufacture as provided herein (alternatively called a bioreactor, a nanobioreactor, a customized cell culture bag or a bioreactor bag), showing trabecular bone structure as recreated using a gelatin sponge, and recreation of blood circulation, including arterioles and sinusoids; and also showing an exemplary ring pump, with in and out ports, including: in the left sub-image, a press point 1, a press point 2 and a press point 3, and a tube for the out port and a tube for the in port, and an eccentric motor with rotation shaft, pump base and ring, where the press point of the tube moves from 1, 2 to 3 (press point 1, a press point 2 and a press point 3) to (cause by) rotations of the eccentric rotor; illustrated is an exemplary Takasago 2 ml/min peristaltic pump in a two-port exemplary system.

FIG. 3 schematically illustrates processing and isolating CD34+ cells from primary human bone marrow for use in an exemplary product of manufacture as provided herein (alternatively called a bioreactor, a nanobioreactor, a customized cell culture bag or a bioreactor bag), where donor bone marrow is centrifuged to isolate mononuclear cell via use of a density gradient or isopycnic centrifugation fluid such as, for example, FICOLL-PAQUE PLUS™ (Cytiva, Fisher Scientific, Thermo Fisher), where density gradient centrifugation separates whole blood, or bone marrow, into: an upper layer comprising plasma; a lower layer comprising lymphocytes, monocytes and platelets; a lower layer comprising the FICOLL-PAQUE PLUS™, and a bottom layer comprising granulocytes and erythrocytes; and also showing use of magnetic labeling with microbeads to isolate CD34+ stromal cells and CD34+ hematopoietic stem and progenitor cells (HSPCs).

FIG. 4 schematically illustrates assembling an exemplary product of manufacture as provided herein (alternatively called a bioreactor, a nanobioreactor, a customized cell culture bag or a bioreactor bag) (in this illustration, an exemplary two-port bag is used) to include stromal cell support and HSPCs; and this exemplary system design includes lentiviral transduction of target HSPCs with a fluorescent reporter to demonstrate live cell imaging capabilities.

FIG. 5 illustrates an exemplary product of manufacture as provided herein (alternatively called a bioreactor, a nanobioreactor, a customized cell culture bag or a bioreactor bag) (in this illustration, an exemplary two-port bag is used) to support long-term cultures of primary hematopoietic stem and progenitor cells in outer space.

FIG. 6 graphically illustrates a flow cytometry analysis of cells returned from an orbital space mission (SpX-CRS24 mission), and the data confirms the ability to culture and maintain primary human hematopoietic stem and progenitor cell viability in an exemplary product of manufacture as provided herein (or nanobioreactor) in space; analysis done with a flow cytometer FORTESSA X-20™ (BD Biosciences) on 50,000 cells, a DAPI (or 4,6-diamidino-2-phenylindole) stain, a marker for membrane viability, and thus for cell viability, was used.

FIG. 7 graphically illustrates a flow cytometry analysis of CD34+ cells returned from an orbital space mission (SpX-CRS24 mission), and the data confirms ability to culture primary human hematopoietic stem and progenitor cells in an exemplary product of manufacture as provided herein (or nanobioreactor) in space; analysis done with a flow cytometer FORTESSA X-20™ (BD Biosciences) on 50,000 cells; a DAPI stain was used.

FIG. 8 illustrates images of fluorescently labeled cells taken during an orbital space mission (SpX-CRS24 mission), and the data confirms the ability to image live cells in an exemplary product of manufacture as provided herein (or nanobioreactor) in space; hematopoietic stem and progenitor cells (HSPCs) were lentivirally transduced with a lentiviral bicistronic fluorescent, ubiquitination-based cell cycle indicator reporter, or a FUCCI2BL™ cell cycle reporter, and images taken with a high resolution live cell imaging scope (or an ETALUMA™, Carlsbad, CA, scope) at 20×; images of cells grown in regular lab conditions (the “ground” upper row of images) are compared to images of cells grown during the space mission (the “flight” lower row of images).

FIG. 9 graphically illustrates a fluorescent quantification of a lentiviral bicistronic fluorescent, ubiquitination-based cell cycle indicator reporter (FUCCI2BL™)-labeled HSPCs during a 34-day space mission; quantification measured using a software capable of 3D analysis of fluorescence images (VOLOCITY™, Ontario, Canada) and graphed in a data analysis software (PRISM™, GraphPad Software, San Diego, CA); mVenus, a basic, constitutively fluorescent, yellow fluorescent protein; and, MCherry, a basic, constitutively fluorescent, red fluorescent protein, were also used; and, cells grown in regular lab conditions (the “ground” graphic) are compared to cells grown during the space mission (the “flight” graphic).

FIG. 10A illustrates a schematic of a post-space mission colony assay where post space mission cells are plated (at 10,000, or 10K, cells/ml), with a two week incubation, followed by a survival (viability) assay; and cells are recovered, mixed and replated at approximately 1000 cells per ml, followed by a two week incubation, followed by a self-renewal assay, and

FIG. 10B graphically illustrates the data from that assay, the data confirming the ability to use, or the efficacy of, an exemplary product of manufacture as provided herein (or nanobioreactor) to maintain long-term cell cultures; in FIG. 10B left image primary cell colonies 2 weeks post plating are analyzed, and in FIG. 10B right image secondary cell colonies 4 weeks post plating are analyzed; and, cells grown in regular lab conditions (the “ground” graphic) are compared to cells grown during the space mission (the “flight” graphic); granulocyte-macrophage progenitor (CFU-GM) cells, multilineage cells, and total cell survival numbers are measured.

FIG. 11 illustrates an assay that provide a cytokine profile of hematopoietic stem cell fitness after exposure to low earth orbit; analysis was done on cell-free supernatants (from cell cultures grown in an exemplary product of manufacture as provided herein (or nanobioreactor)) collected after payload splashdown; cells grown in regular lab conditions (the “ground” plates) are compared to cells grown during the space mission (the “flight” plates); data shows that genes upregulated in space flight are IGFBP-2, PDGF-BB, IL-6, IL1a and OPG (or TNFRSF118); and in contrast genes upregulated during ground culture are TIMP-1, TIMP-2 and IL6-R.

FIG. 12A-C illustrate the result of whole genome and whole transcriptome sequencing analyses, which data demonstrates the ability to culture HSPCs in space using an exemplary product of manufacture as provided herein (or nanobioreactor), and returning viable cells for isolation of bulk DNA and RNA;

FIG. 12A graphically illustrates telomere length of flight (space mission) versus ground sample cells, as measured using a computational tool for estimating the average telomere length (TL) for a paired end, whole genome sequencing (WGS) sample, or TELOMERECAT™ (see, for example, Farmery et al, Scientific Reports, vol 8, Article number: 1300 (2018)) and a software for the detailed characterization of telomere maintenance mechanism footprints in the genome, or TELOMEREHUNTER™ (see, for example, Feuerbach et al, BMC Bioinformatics vol 20, Article number: 272 (2019));

FIG. 12B graphically illustrates normalized singleton counts using different probes in flight (space mission) versus ground sample cells, the nucleic acid probes are: TTTGGG, TGAGGG, TTGGGG, TGAGGG, TAAGGG, CTAGGG, TCAGGG, TCCGGG, ATAGGG, CATGGG; and,

FIG. 12C graphically illustrates a distribution summary of flight versus ground sample cells.

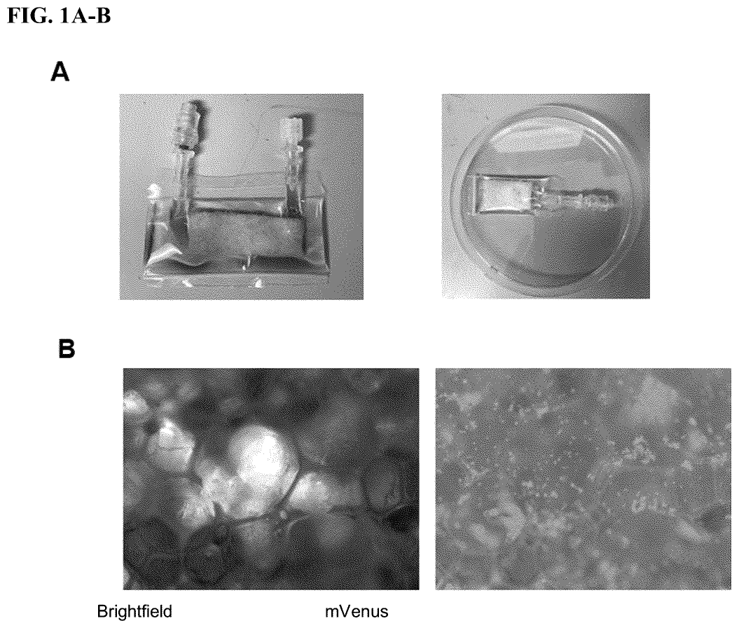

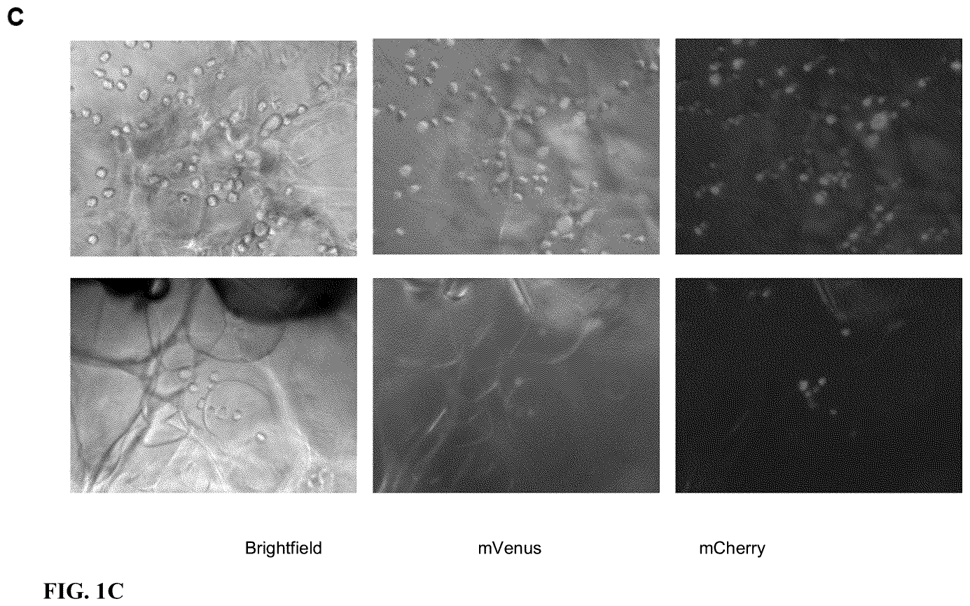

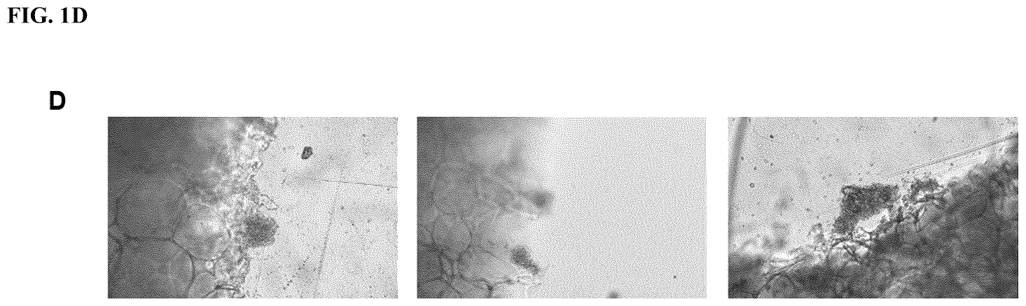

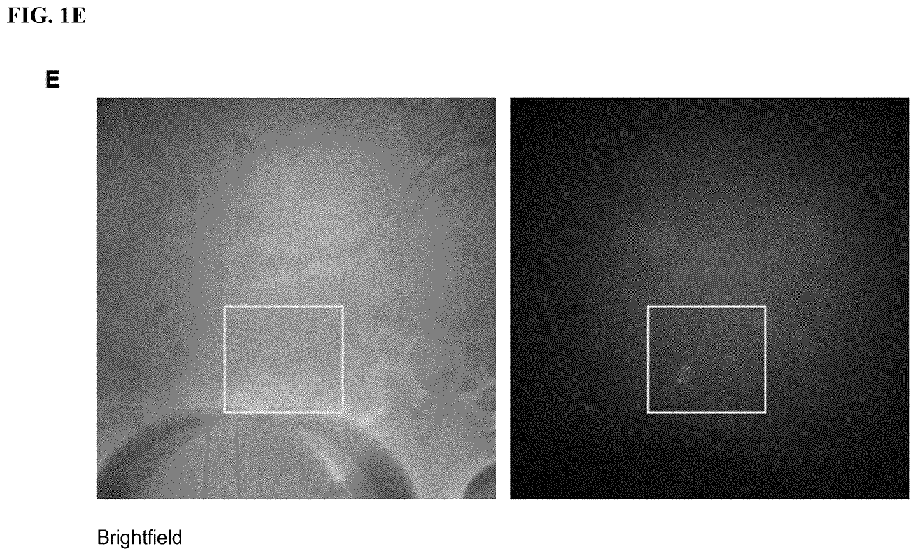

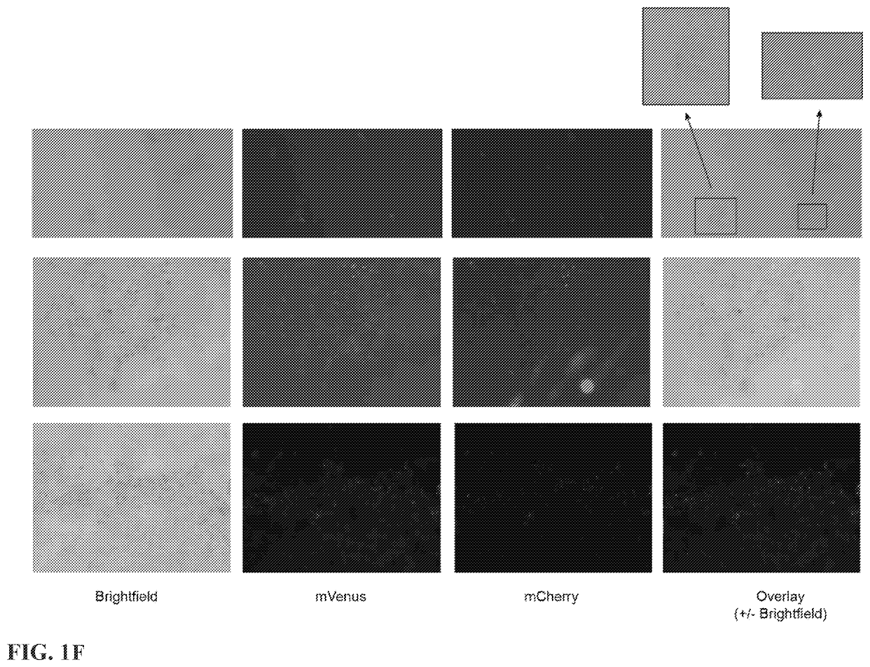

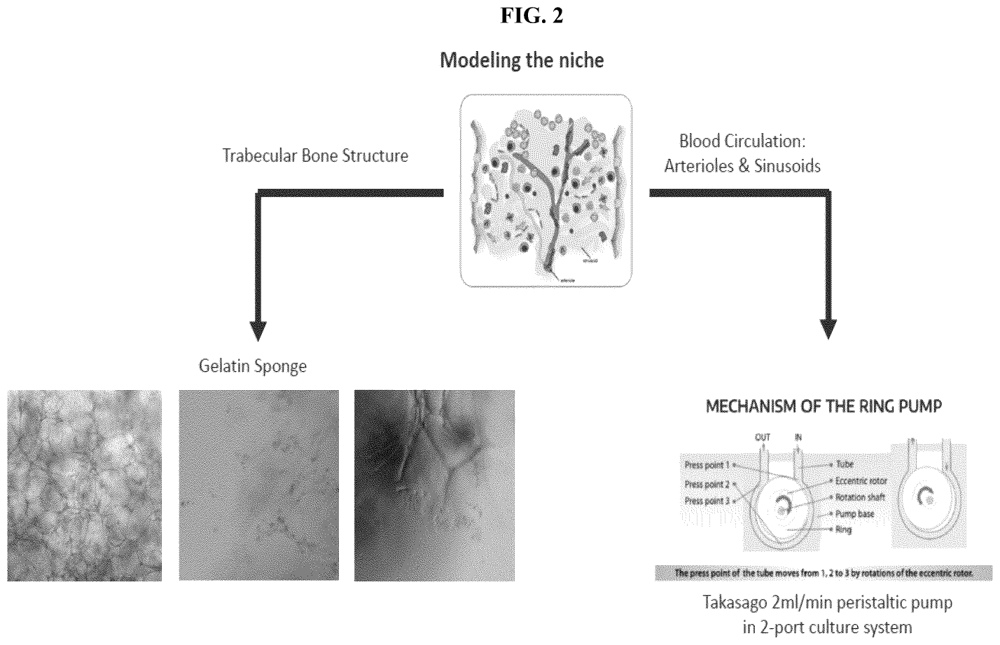

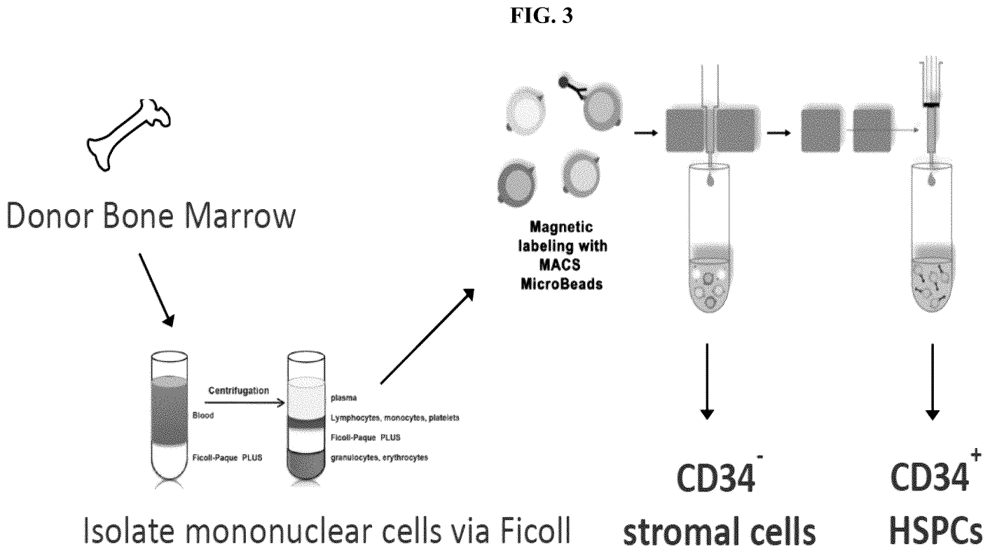

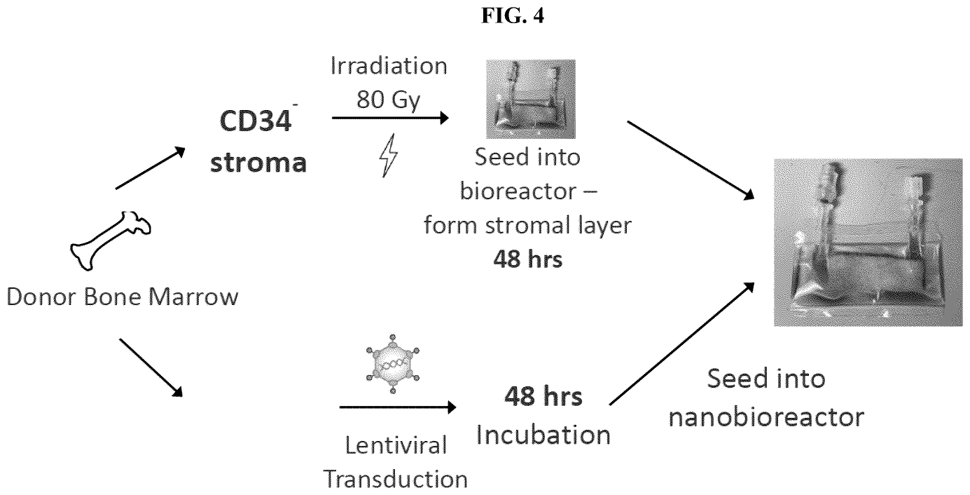

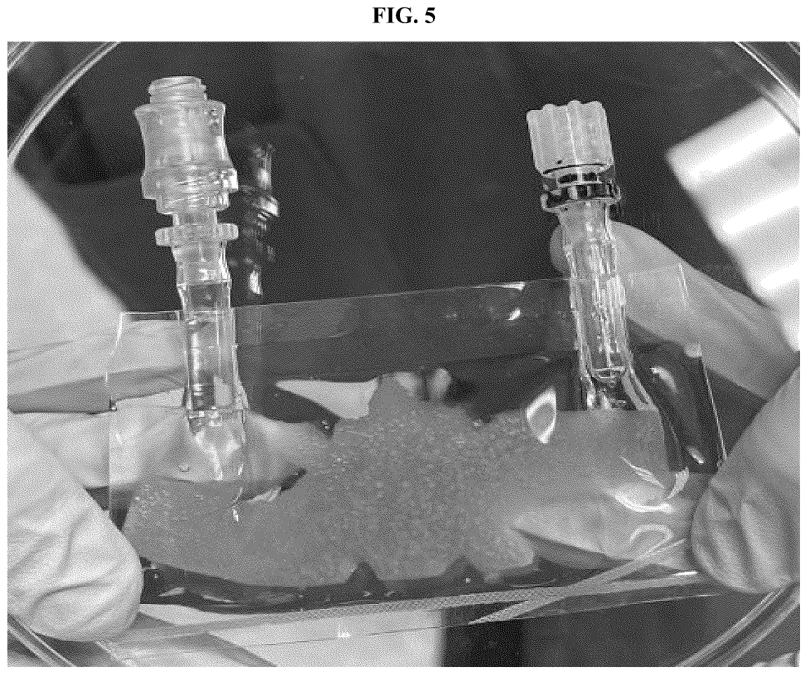

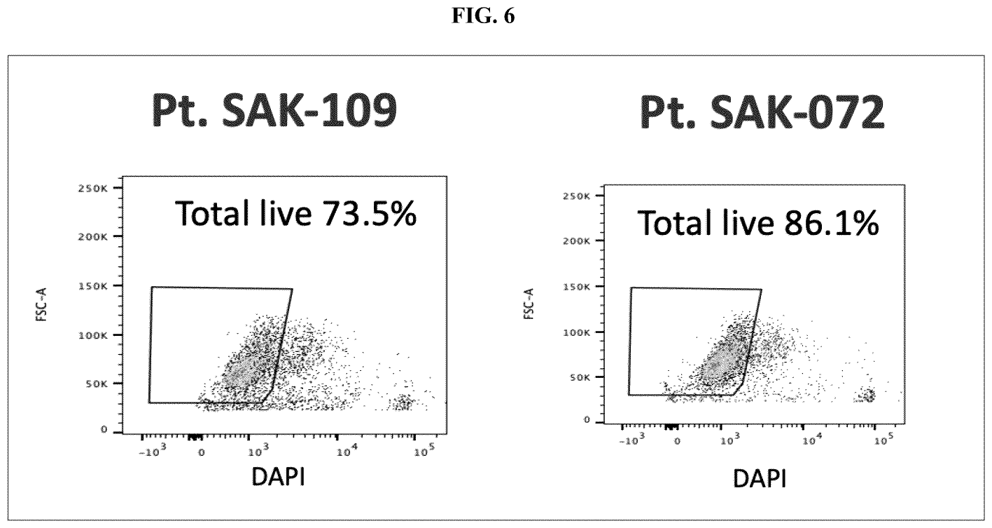

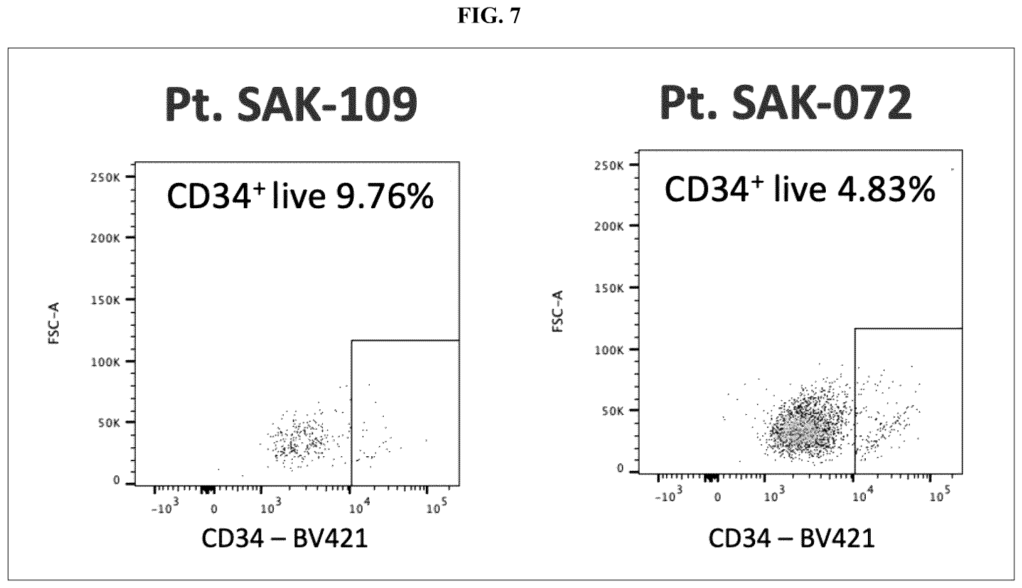

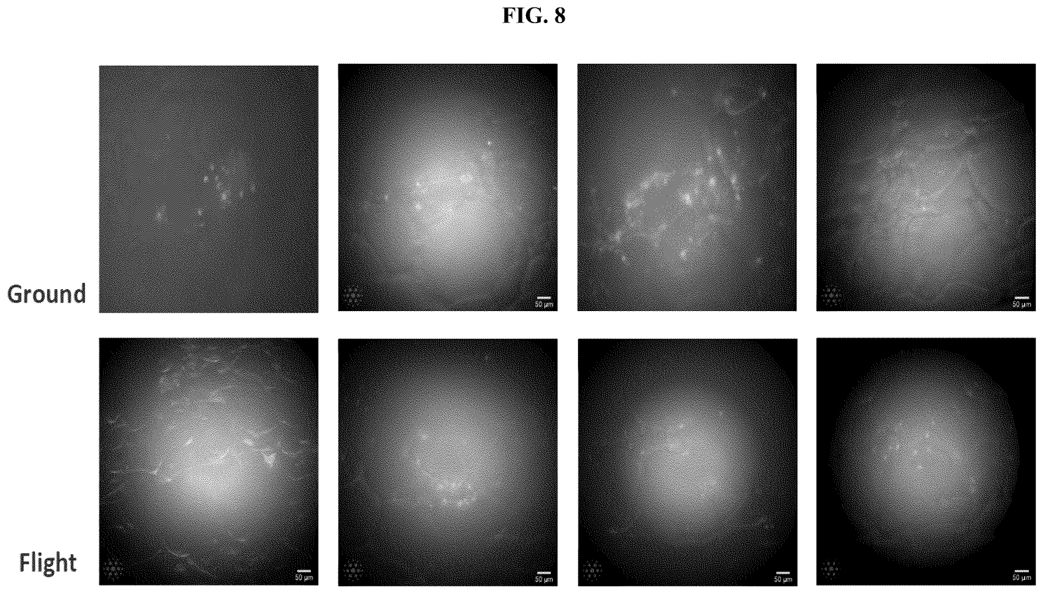

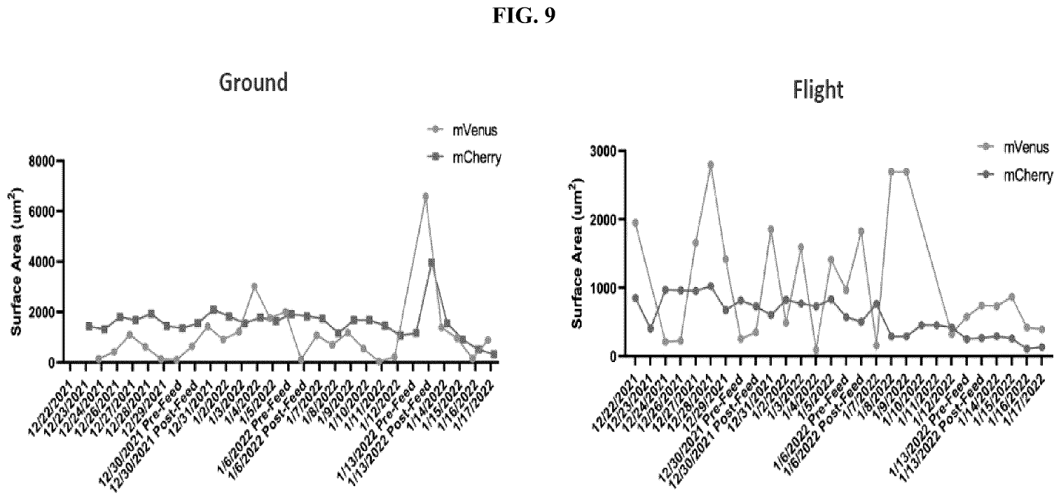

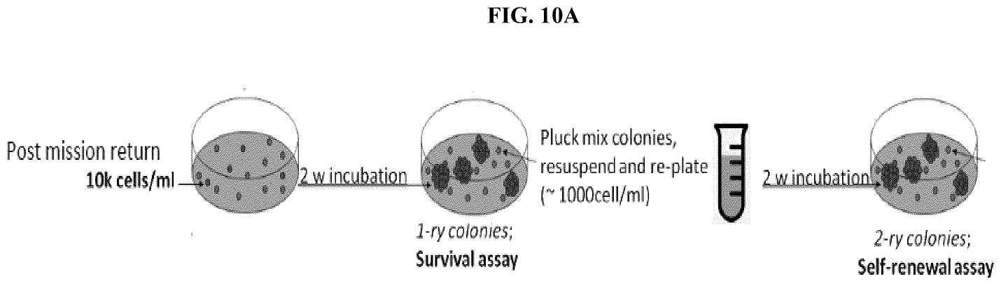

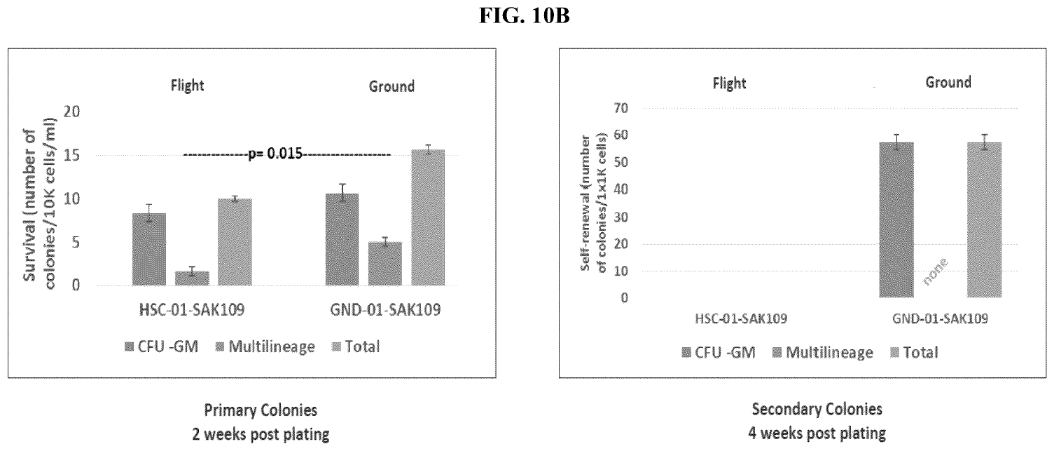

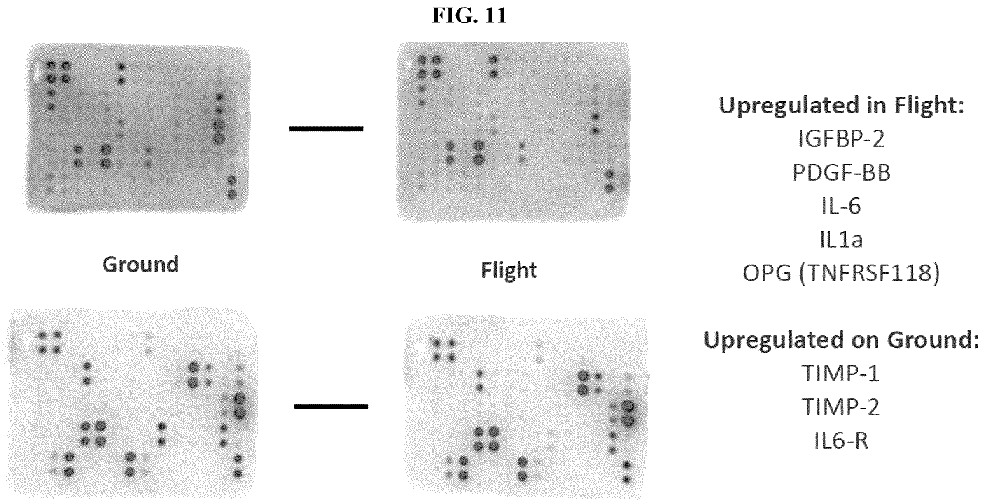

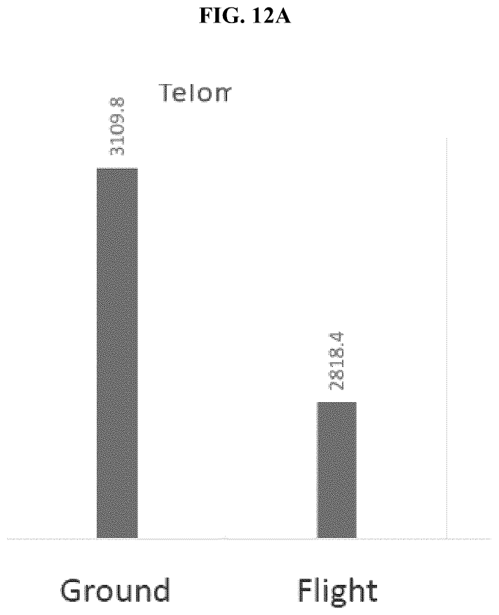

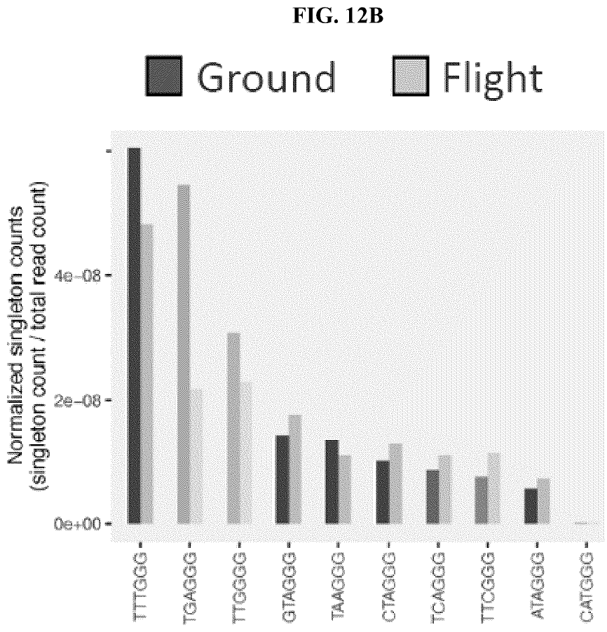

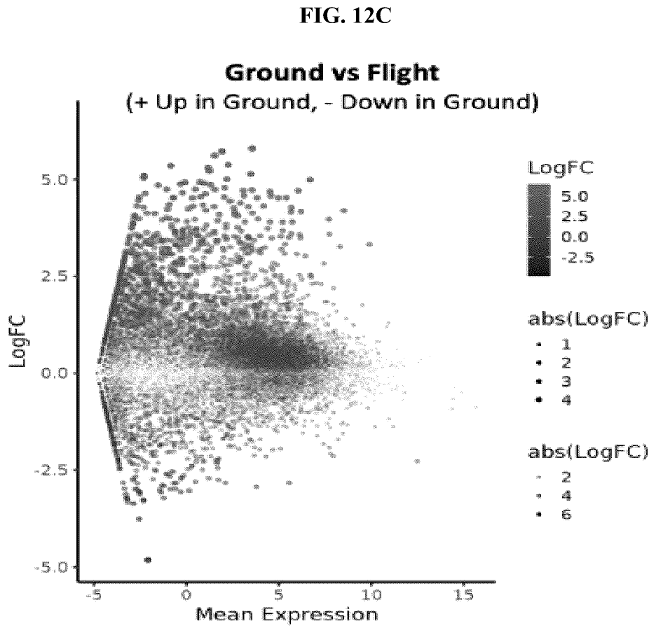

Like reference symbols in the various drawings indicate like elements.

### DETAILED DESCRIPTION

In alternative embodiments, provided are products of manufacture in the form of a bioreactor, a nanobioreactor, a bag or a customized cell culture bag, and kits comprising them, and methods for making and using them. In alternative embodiments, the products of manufacture as provided herein, including the bioreactor, nanobioreactor, bags, customized cell culture bag and kits as provided herein, are used to and are capable of culturing, supporting the culture of, maintaining in culture, and/or differentiating cells, for example, blood cells, stem cells and/or hematopoietic stem cells, including human hematopoietic stem cells, which can be isolated from bone marrow or blood donors. In alternative embodiments, the products of manufacture as provided herein, including the bioreactor, nanobioreactor, bag, customized cell culture bag and kits as provided herein, are used to and are capable of supporting or maintaining (for example, maintaining the cell viability of) a primary human hematopoietic stem cell culture.

In alternative embodiments, the products of manufacture as provided herein, including the bioreactor, nanobioreactor, bag, customized cell culture bag and kits as provided herein, recreate or simulate, or substantially simulate or are substantially similar to, a bone marrow niche such as a human bone marrow niche, and wherein in alternative embodiments, this environment is generated or created by use or inclusion of a sponge matrix (or a sponge-like material) or equivalents in the product of manufacture (or the bioreactor, nanobioreactor, bag or customized cell culture bag) to simulate or to imitate (or substantially simulate or to imitate) a trabecular bone structure, and wherein the sponge or sponge-like material or equivalent is isolated in, or located within, a gas-permeable container or gas-permeable cell culture bag, thereby allowing for three-dimensional cell culture (including for example, growth or differentiation or maintenance) of hematopoietic stem cells, including human, hematopoietic stem cells, including for example CD34+ donor-derived human hematopoietic cells (HSCs). In alternative embodiments, a CD34+-fraction of the same donor bone marrow is seeded into (or added to) the product of manufacture (or the bioreactor, nanobioreactor, bag or customized cell culture bag) together with the HSCs to produce cytokines and growth factors, thus closely mimicking bone marrow niche and/or trabecular bone structure conditions.

In alternative embodiments, the products of manufacture as provided herein, including the bioreactor, nanobioreactor, bag, customized cell culture bag and kits as provided herein, recreate (or substantially simulate or reproduce) a human bone marrow niche by comprising use of a sponge or sponge-like matrix or equivalent to imitate a trabecular bone structure, wherein the sponge or sponge-like matrix or equivalent is inserted in or is located within a gas-permeable container such as a gas-permeable bag such as a gas-permeable cell culture bag, thereby allowing for three-dimensional cell culture of, for example, stem cells, or hematopoietic cells (HSCs), or CD34+ donor-derived human hematopoietic cells (HSCs). In alternative embodiments, the CD34⁻ fraction of the same donor bone marrow is seeded into the product of manufacture (for example, bioreactor or bag) together with the HSCs, thereby producing cytokines and growth factors to closely or substantially mimic or reproduce a niche condition, for example, a bone marrow niche condition or trabecular bone structure.

In alternative embodiments, the products of manufacture as provided herein, including the bioreactor, nanobioreactor, bag, customized cell culture bag and kits as provided herein, are designed with the purpose of supporting or maintaining primary human hematopoietic stem cell cultures in a low gravity environment, for example, in a low (space) orbit environment, for example, on orbit aboard the International Space Station (ISS). This design, however, can be used to recreate and simulate the hematopoietic stem cell niche for any other purpose and under any other circumstances as well.

In alternative embodiments, the products of manufacture as provided herein, including the bioreactor, nanobioreactor, bag, customized cell culture bag and kits as provided herein can support maintenance, development, growth and/or differentiation of organoids, including for example an organoid derived from (or grown from) stem cells (such as human hematopoietic cells (HSCs)) and/or cancer stem cells. In alternative embodiments, this is facilitated by adjusting or augmenting a sponge or sponge-like matrix or equivalent by addition of a composition facilitating the support, maintenance, development, growth and/or differentiation of stem cells, or addition of a cell culture media, for example, a cell culture media that can facilitate support, maintain, develop, grow and/or differentiate stem cells. In alternative embodiments, the sponge or sponge-like matrix or equivalents can also comprise a demineralized cancellous bone matrix sponge, or demineralized bone matrix components comprising for example OSTEOSPONGE™ (Xtant Medical). In alternative embodiments, the sponge or sponge-like matrix or equivalents can also comprise: an absorbable gelatin sponge such as an absorbable human or porcine gelatin sponge, a solubilized or reconstituted basement membrane matrix or a solubilized or reconstituted laminin/collagen IV-rich basement membrane extracellular matrix (for example, MATRIGEL™ (Corning Life Sciences) or GELTREX™ (ThermoFisher Scientific) or a compressed sponge with adsorbable gelatin such as GELFOAM™ (Pfizer) or a hydrogel-based macroporous sponge or porous hydroxipropylcellulose (HPC) scaffold such as CELLUSPONGE™, CELLUSPONGE-GAL™, or CELLUSPONGE-COL™ (Bio-Bybios)),

In alternative embodiments, any cell culture media capable of supporting hematopoietic stem cell cultures can be used.

In alternative embodiments, any gas permeable bag or equivalent container can be used, or materials similar to know gas permeable containers can be used, for example, a product of manufacture as provided herein can comprise use of a gas permeable material or membranes comprising fluorinated ethylene propylene (FEP) copolymers, for example, as found in: VUELIFE 2 PF-0290™ (American Fluoroseal Corporation, Gaithersburg, Md) and PERMALIFE™ (OriGen Biomedical, Austin, Tex); and/or comprising polyolefins, for example, as in culture bags from LIFECELL™ (3-L bag, Baxter, Deerfield, Ill) made of polyolefin blends, or CULTILIFE™ (Takara) cell-culture bags; or MACS™ culture bags (Miltenyi Biotec, Bergisch Gladbach, Germany).

In alternative embodiments, a gas permeable material or membranes used to made products of manufacture as provided herein comprise: nonporous polystyrene, microporous polyolefin, such as POLYFLEX® (Plastic Suppliers), microporous high density polyethylene (HDPE), such as TYVEK®, or TYVEK® 1073 (DuPont), microporous polypropylene, microporous polyvinylidene fluoride, track-etched polycarbonate (optionally of small diameter, non-wet in the liquid phase), hydrophobically treated nylon, polyurethane, microporous polyester having hydrophobic pores, microporous inorganic polymer and nonporous silicone rubber, any inorganic polymer, co-extruded polystyrene and non-porous polyethylene or styrene such as butadiene-styrene, or ethyl vinyl acetate, or styrene-butadiene-styrene (SBS/EVA/SBS) three-layer coextruded film, or styrene-butadiene-styrene/polyethylene (SBS/PE) two-layer coextruded film.

In alternative embodiments, gas-permeable (and optionally, liquid impermeable or liquid impervious) membranes used are about 0.0051 cm (0.002 inch) in thickness.

In alternative embodiments, gas-permeable (and optionally, liquid impermeable or liquid impervious) membranes used comprise a microporous membrane coated with a thin layer of silicone, the permeability of silicone makes it particularly suitable for gas exchange, and optionally the surface coating can be a thin layer of poly[1-(trimethylsilyl)-1-propyne] (PTMSP), which is known to have very high permeability for gases.

In alternative embodiments, gas-permeable (and optionally, liquid impermeable or liquid impervious) membranes used comprise hydrophobic microporous hollow fiber membranes for degassing applications to remove oxygen, carbon dioxide, and other gases from the culture media, water and/or other liquids. In alternative embodiments, a commercial membrane module such as LIQUI-CEL® membrane contactor (Membrana, Charlotte, N.C.) is used, which can comprise polypropylene (PP) ×40 or ×50 hollow fibers. In alternative embodiments, a microporous PP hollow fiber membrane (for example, CELGARD®) is used for oxygenation applications. In alternative embodiments, membrane modules suitable for large scale industrial applications that have large membrane surface areas (for example, 220 m² active membrane surface area) are used, for example, LIQUI-CEL®.

In alternative embodiments, gas-permeable (and optionally, liquid impermeable or liquid impervious) membranes used comprise poly (vinylidene fluoride) (PVDF), polyethylene (PE), PP, poly (vinyl chloride) (PVC), or other polymeric materials, and optionally a pore size is in the range of 0.03 to 0.4 μm, and optionally a hollow fiber is used, for example, having an outer diameter is 0.5 to 2.8 mm and inner diameter 0.3 to 1.2 mm.

Because low Earth orbit (LEO) can be used as a model to study inflammation, aging, and malignant transformation in stem cells, we designed and developed a bioreactor system, as provided herein, for supporting cell cultures of donor-derived human HSPCs in LEO. In alternative embodiments, a sponge matrix, continuous flow pumps and stromal cells are used, and this system, or product of manufacture as provided herein, can be used to model the bone marrow niche. Viability assessment via flow cytometry demonstrates our system's (our product of manufacture's, as provided herein) ability to maintain stem cell fitness over 6 weeks. Cell cycle tracking shows a sharp decrease in hematopoietic stem and progenitor cells in the resting (G0/G1) phase of the cell cycle. During spaceflight compared to ground, in vitro colony assays post-flight confirm decreased survival and loss of self-renewal capacity in stem cells returning from spaceflight. These data gained from the LEO SpX-24 mission demonstrate a clear trend towards hematopoietic stem cell exhaustion and reduced “stemness” after 30-day exposure to LEO.

### Culturing Conditions

In alternative embodiments, the products of manufacture and kits as provided herein comprising cells are cultured at cell culture growth compatible temperatures and environmental conditions, for example, as described by Boudewijn Van Der Sanden, J Cell Biochem. 2010 November; 111 (4): 801-807; or Borowski, et al, Basic pluripotent stem cell culture protocols, StemBook, Cambridge (MA): Harvard Stem Cell Institute; 2008.

In alternative embodiments, the products of manufacture and kits comprise use of culture media compatible with stem cells, cultured cancer cells or organoids, which can be supplemented with growth factors or serum, see for example: Lee et al, Cell Biol Int. 2022 January; 46(1): 139-147, Epub 2021 Nov. 27; Zhang J Tissue Eng 2020 Jun. 24; 11.

In alternative embodiments, the products of manufacture further comprise a pump, for example, a peristaltic pump, for example, a micro-peristaltic pump, for circulating liquid such as culture media inside the container, enclosure or bag, where the pump is operably connected to the in port and the out port.

In alternative embodiments, the products of manufacture and kits further comprise sensors for measuring the flow of liquid from the pump or pumps, or for measuring a culture environment parameter, such as measuring the temperature and/or pressure of the environment in the product of manufacture, or the amount of oxygen or carbon dioxide in the product of manufacture, or the pH of the product of manufacture, or the viscosity or turbidity of fluid in the product of manufacture. In alternative embodiments, the output of the sensors, and/or the pumps, are communicated with a remote device, which can be read by a user, where the device allows the user to feedback or adjust the operation of the pump, or adjust another parameter, for example, pH, temperature, gas volumes (for example, amount of oxygen) and/or pressure.

In alternative embodiments, the rate of oxygen consumption by cells in the product of manufacture is balanced by adjusting the amount of oxygen transported through the membrane or the amount of oxygen transported into the product of manufacture by flowing culture or biological fluids in the product of manufacture. The kinetics of oxygen consumption by the cell is given by the oxygen uptake rate (OUR). The oxygen uptake rate can be expressed by the Michaelis-Menten kinetics, expressed as the number of moles of oxygen consumed per unit time per cell multiplied by the number of cells. The rate of oxygen transport through the membrane is the volume flow of oxygen per unit partial pressure difference (expressed as the volume of oxygen per unit area per unit time per unit partial pressure difference), the partial pressure difference inside and outside the membrane, and used. It is given as the product of the projected area of the film. The rate of oxygen transport by the culture or biological fluid depends on the diffusion rate of oxygen in the biological fluid, the oxygen concentration gradient along the direction perpendicular to the membrane on the membrane surface of the part where the cells are cultured, and the membrane composition.

### Cellular Sensors and Reporter Vehicles or Vectors

In alternative embodiments, cell in the products of manufacture and kits further comprise molecular sensors or reporters, for example, bioluminescent and/or fluorescent sensors or reporters, including vectors, plasmids or viral vectors, such as bioluminescent and fluorescent lentiviral or adenoviral reporter vectors; wherein the molecular sensors are capable of measuring and/or quantifying one or more cell functions, for example, capable of measuring and/or quantifying normal, pre-cancer and/or cancer cell or stem cell function and/or viability.

In alternative embodiments, any molecular sensor can be used, for example, any vector, any viral vector, such as for example a lentiviral vector, including for example a cell cycle reporter such as a FUCCI2BL cell cycle reporter, a self-renewal reporter such as an ADAR1 (adenosine deaminase acting on RNA-1)-nanoluciferase-GFP (green fluorescent protein) self-renewal reporter, and/or a splicing reporter such as a monomeric red fluorescent protein (mRFP) reporter, or an RFP/GFP splicing reporter.

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1

This example describes exemplary protocols for making and using products of manufacture, for example, bioreactors, as provided herein.

### Materials

ULine Tabletop IMPULSE HEATSEALER H-163™
ORIGEN PERMALIFE™ gas-permeable cell culture bags
ETHICON SURGIFOAM™
absorbable porcine gelatin sponge
Sterile scissors, tweezers,
scalpel
Sterile luer lock syringes
   Sterilize all non-sterile equipment by autoclaving. Work in a sterile environment, such as a biosafety cabinet and spray and wipe all materials with 70% EtOH prior to use.
1) Use scalpel to cut sponge into desired shape and size.
2) Pre-soak sponge in media desired to use for cell culture to get rid of air bubbles inside the sponge. Consider degassing media depending on downstream application.
3) Use scissors to cut OriGen cell culture bag open along the side facing away from the port(s).
4) Use tweezers to insert pre-soaked sponge into bag. Be careful not to wet the edges of the bag as it may interfere with re-sealing.
5) Use heatsealer to re-seal the bag with 9 consecutive impulses on the highest heat setting. Wait approx. 2 seconds in between impulses.
6) Leave bag on heatsealer for 5 min to cool down before peeling it off to avoid ripping the plastic of the bag and introducing a leak.
7) Carefully peel bioreactor bag off the heatsealer and test re-seal with tweezers. If re-seal is satisfactory and no hole is found, move on to step 8. If not, repeat from step 5.
8) Fill syringe with desired cell culture media and fill bioreactor bag about halfway (test volumes depending on size of cell culture bag used!).
9) Fill syringe with cells at desired density and seed bioreactor bag. Fill syringe with second half of media volume and fill bioreactor bag to flush cells out of port into bag and sponge.
10) Let cells settle for 8-24 hrs and proceed with downstream application.

Because low Earth orbit (LEO) can be used as a model to study inflammation, aging, and malignant transformation in stem cells, we designed and developed a bioreactor system, as provided herein, for supporting cell cultures of donor-derived human HSPCs in LEO. In alternative embodiments, a sponge matrix, continuous flow pumps and stromal cells are used, and this system, or product of manufacture as provided herein, can be used to model the bone marrow niche. Viability assessment via flow cytometry demonstrates our system's (our product of manufacture's, as provided herein) ability to maintain stem cell fitness over 6 weeks. Cell cycle tracking shows a sharp decrease in hematopoietic stem and progenitor cells in the resting (G0/G1) phase of the cell cycle. During spaceflight compared to ground, in vitro colony assays post-flight confirm decreased survival and loss of self-renewal capacity in stem cells returning from spaceflight. These data gained from the LEO SpX-24 mission demonstrate a clear trend towards hematopoietic stem cell exhaustion and reduced “stemness” after 30-day exposure to LEO.

A number of embodiments of the invention have been described. Nevertheless, it can be understood that various modifications may be made without departing from the spirit and scope of the invention. Accordingly, other embodiments are within the scope of the following claims.

1. A product of manufacture for primary human hematopoietic stem cell culture and/or maintenance, comprising:
a container, enclosure or bag having gas-permeable walls or sides, and a sponge matrix or a sponge-like material contained within the container, enclosure or bag,
wherein the sponge matrix or sponge-like material is infused with a cell culture media and a mixture of cells comprising stem cells and/or bone marrow matrix cells,
and the container, enclosure or bag interior is sterile,
and the container, enclosure or bag comprises at least one liquid or cell input port.

2. The product of manufacture of claim 1, wherein the container, enclosure or bag is fabricated as a gas-permeable cell culture environment.

3. The product of manufacture of claim 1, wherein the container, enclosure or bag comprises at least two liquid or cell input ports, one out port and one in port.

4. The product of manufacture of claim 3, wherein a micro-peristaltic pump for circulating liquid inside the container, enclosure or bag is operably connected to the in port and the out port.

5. The product of manufacture of claim 1, wherein the mixture of cells comprises stem cells, bone marrow matrix cells, or cancer stem cells, or human cancer stem cells, or organoid cells, or comprising cells from malignant organoids representing solid tumors.

6. The product of manufacture of claim 1, wherein the mixture of cells comprises CD34+ donor-derived human hematopoietic cells (HSCs), or human CD34+ donor-derived human hematopoietic cells (HSCs).

7. The product of manufacture of claim 1, wherein the sponge matrix or sponge-like material comprises:
an absorbable gelatin sponge, and/or
a compressed sponge with adsorbable gelatin,

8. The product of manufacture of claim 7, wherein the sponge or sponge-like material further comprises: a demineralized cancellous bone matrix sponge, or demineralized bone matrix components.

9. The product of manufacture of any of claims claim 1, wherein between about 10⁶ to about 10¹⁰ cells are placed into, or cultured in, the product of manufacture.

10. The product of manufacture of claim 1, further comprising a detectable vector or reporter, optionally comprising a bioluminescent and/or a fluorescent vector, optionally comprising a bioluminescent and/or a fluorescent lentiviral reporter vector, wherein optionally the bioluminescent and/or a fluorescent vector is capable of quantifying cells or cell function, optionally capable of quantifying normal, pre-cancer and/or cancer stem cell function, optionally the detectable vector or report comprises a FUCCI2BL cell cycle reporter, an ADAR1-nanoluciferase-GFP reporter self-renewal reporter and/or an RFP/GFP splicing reporter.

11. A method for culturing or maintaining or differentiating a plurality of stem cells comprising placing into or culturing the plurality of stem cells in a product of manufacture of claim 1.

12. A method for producing an organoid, optionally an malignant organoid representing a solid tumor, optionally breast cancer solid tumor comprising incubating in a product of manufacture of claim 1 a plurality of stem cells, optionally further comprising cytokines capable of differentiating the stem cells.

13. A kit comprising a product of manufacture of claim 1, optionally further comprising instructions for practicing a method of any of the preceding claims.

14-15. (canceled)

16. The product of manufacture of claim 1, wherein the container, enclosure or bag having gas-permeable walls or sides is liquid impermeable or liquid impervious.

17. The product of manufacture of claim 5, wherein the mixture of cells comprises stem cells, or human stem cells.

18. The product of manufacture of claim 7, wherein the absorbable gelatin sponge comprises an absorbable human or porcine gelatin sponge.

19. The product of manufacture of claim 1, wherein the sponge matrix or sponge-like material comprises a solubilized or reconstituted basement membrane matrix.

20. The product of manufacture of claim 1, wherein the sponge matrix or sponge-like material comprises a solubilized or reconstituted laminin/collagen IV-rich basement membrane extracellular matrix.

21. The product of manufacture of claim 1, wherein the sponge matrix or sponge-like material comprises a hydrogel-based macroporous sponge or porous hydroxipropylcellulose (HPC) scaffold.

22. The product of manufacture of claim 5, wherein the bone marrow matrix cells comprise human bone marrow matrix cells.
